(19) United States
(12) Patent Application Publication
(10) Pub. No.: US 2018/0199830 A1
(43) Pub. Date: Jul. 19, 2018

(54) WEARABLE TONOMETER WITH RESILIENTLY DEFORMABLE PAD

(71) Applicant: Microsoft Technology Licensing, LLC, Redmond, WA (US)

(72) Inventors: Sumit Basu, Redmond, WA (US); Matthew Mickelson, Seattle, WA (US); T. Scott Saponas, Woodinville, WA (US); Sidhant Gupta, Bellevue, WA (US); Ronald E. Paulsen, Woodinville, WA (US)

(73) Assignee: Microsoft Technology Licensing, LLC, Redmond, WA (US)

(21) Appl. No.: 15/406,508

(22) Filed: Jan. 13, 2017

(51) Int. Cl. A61B 5/021; A61B 5/00

(52) U.S. Cl. CPC A61B 5/02108; A61B 5/6831; A61B 5/02141; A61B 5/6843; A61B 5/6824

(57) ABSTRACT

A wearable tonometer is provided, comprising a sensing device. The sensing device may include a pressure sensor configured to measure a pulse pressure wave in an artery of user. The sensing device may include a resiliently deformable pad or pad-cap structure positioned on a sensing surface side of the pressure sensor and configured to contact skin of the user proximate the artery. The wearable tonometer may include a band that holds the sensing device in contact with the skin. In some embodiments, the sensing device may include a rigid internal structure configured to transmit the pulse pressure wave. In some embodiments, the wearable tonometer may include an adjustment mechanism configured to move the sensing device relative to the band. In some embodiments, the wearable tonometer may include a second resiliently deformable pad-cap structure, and a solid plate attached to the resiliently deformable pad-cap structures and the band.

### BACKGROUND

When the left ventricle of the heart compresses and expels blood into the arteries, a pressure wave is created. This wave travels along the arteries in the body, and its shape at any point in the arterial system is affected by the overall blood pressure, the level of vasodilation or vasoconstriction, the state of the endothelial lining of the arteries, the buildup of plaque therein, and the resulting stiffness or compliance of the arteries from the latter to factors. A pressure sensor at a given point in an artery can transduce this pressure as it changes with time and the resulting pulse pressure wave can be recorded.

The pulse pressure wave may be detected invasively or non-invasively. The invasive form of sensing device is a catheter inserted into the artery with a pressure sensor on the tip. Invasive sensing can be used in any artery but is typically used in the descending aorta for so-called “central” measurements. The non-invasive approach is to place a pressure sensor on the surface of the skin over an artery and to applanate the artery, i.e., apply force such that the surface of the artery is at least partial deformed so that the sensor can capture the pressure signal within the artery. The latter is most effective for arteries that are present near the surface of the skin, including the radial (wrist), carotid (neck), and femoral (thigh) arteries. A pressure sensor used to transduce pulse-pressure waves in this way is referred to as a tonometer.

### SUMMARY

A wearable tonometer is provided, comprising a sensing device. The sensing device may include a pressure sensor configured to measure a pulse pressure wave of blood pressure in an artery of a user. The sensing device may further include a resiliently deformable pad or pad-cap structure positioned on a sensing surface side of the pressure sensor and configured to contact skin of the user proximate the artery. The wearable tonometer may further include a band that holds the sensing device in contact with the skin of the user. In some embodiments, the sensing device may further include a rigid internal structure positioned at least partially within the resiliently deformable pad and configured to transmit the pulse pressure wave on its way to the pressure sensor. In some embodiments, the wearable tonometer may further include an adjustment mechanism configured to move the sensing device relative to the band. The adjustment mechanism may be configured to move the sensing device in a plane tangent to the skin of the user while the position of the band fixed. In some embodiments, the wearable tonometer may further include a second resiliently deformable pad-cap structure adjacent to skin of the user, and a solid plate attached to the first resiliently deformable pad-cap structure, the second resiliently deformable pad-cap structure, and the band.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 depicts a wearable tonometer including a sensing device, according to one embodiment of the present disclosure.

FIG. 2 depicts a wearable tonometer with a sensing device including a rigid internal structure, according to one embodiment of the present disclosure.

FIG. 3 depicts structures that may be included in the sensing device of FIGS. 1 and 2, according to one embodiment of the present disclosure.

FIG. 4 depicts a cross-sectional view of a sensing device including a rigid internal structure, according to one embodiment of the present disclosure.

FIG. 5 depicts a cross-sectional view of a sensing device including a pressurized fluid contained within the sensing device, according to one embodiment of the present disclosure.

FIG. 6 depicts a wearable tonometer including an adjustment mechanism, wherein the adjustment mechanism includes biasing members, according to one embodiment of the present disclosure.

FIG. 7 depicts a wearable tonometer including an adjustment mechanism, wherein the adjustment includes screw members, according to one embodiment of the present disclosure.

FIG. 8 depicts a wearable tonometer including an adjustment mechanism, wherein the adjustment mechanism includes a pressurized fluid, according to one embodiment of the present disclosure.

FIG. 9 depicts a wearable tonometer including an adjustment mechanism, wherein the adjustment mechanism includes a cantilever, according to one embodiment of the present disclosure.

FIG. 10 depicts a wearable tonometer including a first sensing device, a second sensing device, and a solid plate, according to one embodiment of the present disclosure.

FIG. 11 schematically depicts a sensory and logic system for use with a wearable tonometer, according to one embodiment of the present disclosure.

### DETAILED DESCRIPTION

The inventors have recognized that traditional tonometers as discussed above require careful placement and manipulation by a skilled technician. Wearable tonometers, which are intended to be easier for patients to place on their own, either tend to be very sensitive to precise placement or uncomfortably bulky. This is because the geometry of the relevant portion of the body, the region where the artery is near the surface of the skin, varies greatly between individuals. In particular, the precise position and depth of any given artery is quite different from patient to patient. A device a small point-like sensor thus requires careful positioning, and thus far the state of the art for avoiding the need for careful positioning is a large, hard, and uncomfortable protrusion that pushes down on the entire region. These shortcomings make existing ambulatory tonometers, which are intended to be worn throughout the day and must be placed correctly by the patient, impractical to use.

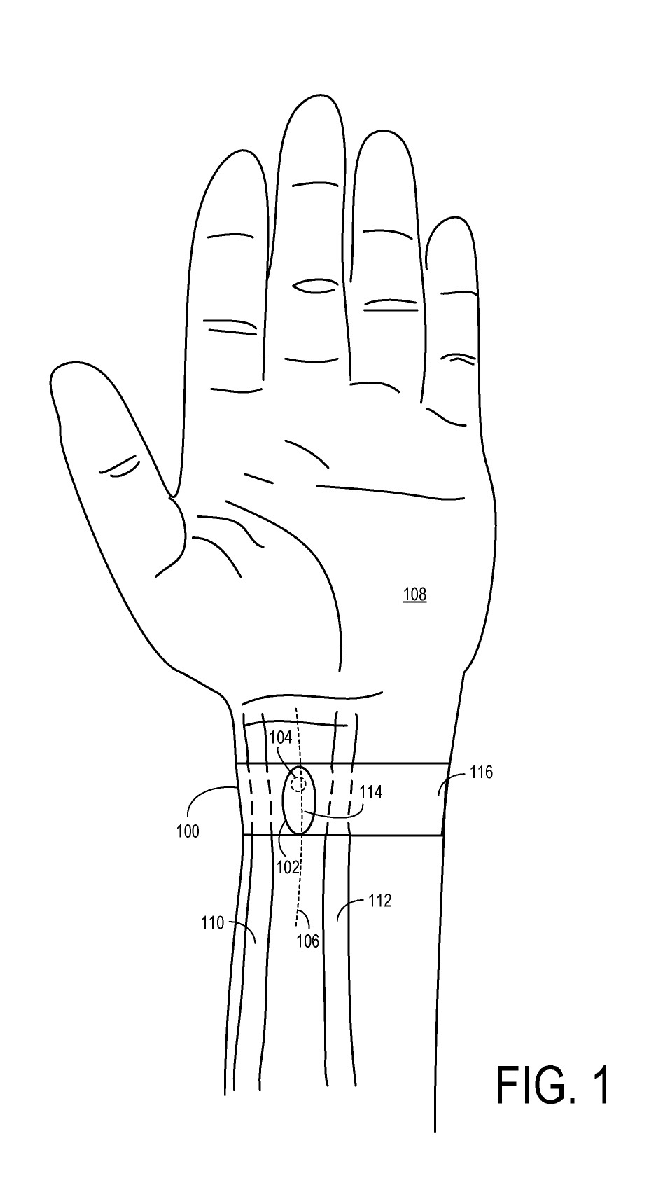

To address these shortcomings, herein is provided a wearable tonometer for ambulatory tonometry which can achieve easy placement without discomfort. FIG. 1 depicts a wearable tonometer 100, according to one embodiment of the present disclosure. The wearable tonometer 100 comprises a sensing device 102, which includes a pressure sensor 104. The pressure sensor 104 is configured to measure, a pulse pressure wave of blood pressure in an ante 106 of a user 108. In the embodiment depicted in FIG. 1, the artery 106 is a radial artery and the sensing device 102 sits between the radius 110 and flexor carpi radialis tendon 112 of the user 108. The wearable tonometer 100 may additionally or instead be configured to measure a pulse pressure wave of blood pressure in other arteries of the user 108.

The wearable tonometer 100 also includes a resiliently deformable pad 114 positioned on a sensing surface side of the pressure sensor 104 and configured to contact skin of the user 108 proximate the artery 106. The resiliently deformable pad 114 may be composed of cloth, foam, elastomeric material, or some other resiliently deformable material or combination of materials. The resiliently deformable pad 114 may applanate the skin of the user 108 proximate the artery 106, applying a force to the skin so that the shape of the skin is slightly deformed. Applanating the skin proximate the artery 106 allows the pressure sensor 104 to detect the pulse pressure wave over a larger surface. Since the precise location of the artery 106 may vary between different users, detecting the pulse pressure wave over a larger surface makes the quality of the measurement made by the wearable tonometer 100 less dependent on the precise placement of the sensing device 102. This allows the user 108 to more easily place the wearable tonometer 100 in a location on the skin at which it will provide an accurate pulse pressure wave measurement. In addition, distributing the applanating force over a larger area may allow the sensing device 102 to continue to detect a pulse pressure wave if the wearable tonometer 100 moves relative to the skin of the user 108. The resiliently deformable pad 114 may also integrate the pulse pressure wave over the body of the sensing device 102, thus concentrating force contributions from a wider area into the pressure sensor 104. By distributing the applanating force applied by the wearable tonometer 100 to the skin, the resiliently deformable pad 114 may also make the wearable tonometer 100 more comfortable for the user 108 to wear.

The wearable tonometer 100 further includes a band 116 that holds the sensing device 102 in contact with the skin of the user 108. In the embodiment shown in FIG. 1, the band 116 is a wrist band. The band 116 may configured to have an adjustable tightness, so as to allow the user 108 to adjust the amount of pressure applied o the skin by the wearable tonometer 100.

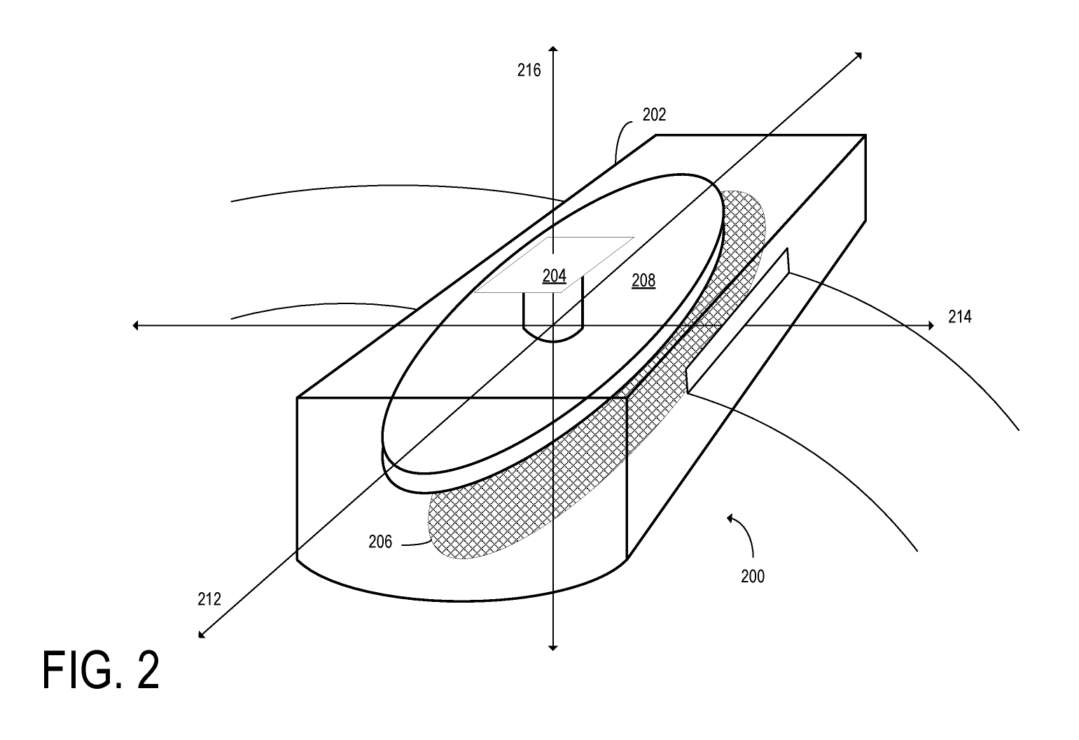

An example embodiment of a wearable tonometer 200 is shown in FIG. 2. The wearable tonometer 200 includes a sensing device 202, including a pulse pressure sensor 204. The pulse pressure sensor 204 may be a piezo-resistive pressure sensor with an electrical resistance that changes in response to pressure, or may be some other variety of pressure sensor. The sensing device 202 further includes a resiliently deformable pad 206. The resiliently deformable pad 206 has a convex skin-contacting surface that is elongated along a longitudinal axis 212. The longitudinal axis 212 may be in line with an artery when the wearable tonometer 200 is worn by a user. The resiliently deformable pad 206 may cover the entire sensing device 202, or alternately may cover only a portion of the sensing device 202 proximate the artery.

According to the embodiment of FIG. 2, the sensing device 202 also includes a rigid internal structure 208 positioned at least partially within the resiliently deformable pail 206. The rigid internal structure 208 is configured to transmit a pulse pressure wave on its way to the pressure sensor 204. The rigid internal structure 208 is composed of a material that is more rigid than the material of which the resiliently deformable pad 206 is composed. The pulse pressure wave may therefore undergo less attenuation when traveling through the rigid internal structure 208 than when traveling through the resiliently deformable pad 206. By including the rigid internal structure 208 in addition to the resiliently deformable pad 206, the sensitivity of the sensing device 202 is increased while retaining the advantages of using a resiliently deformable pad 206.

As shown in FIG. 2, the rigid internal structure 208 may have a flat, plate-like shape that is elongated along the longitudinal axis 212. The rigid internal structure 208 is not limited to having an elliptical perimeter, but may also have a rectangular perimeter, a perimeter shape that includes wing-like protrusions, or some other perimeter shape.

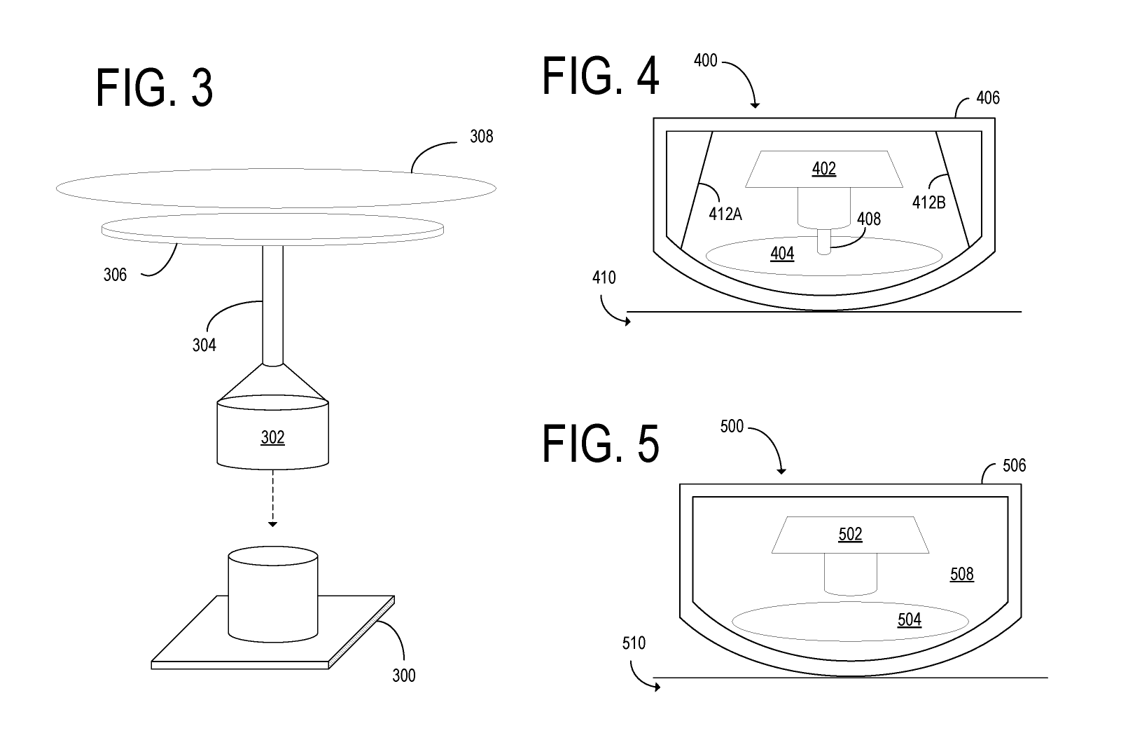

FIG. 3 depicts structures that may be included in the sensing device of FIGS. 1 and 2. The pressure sensor 300 may be covered by a resiliently deformable cap 302 on the side of the pressure sensor 300 proximate the artery of the user. The resiliently deformable cap 302 may protect the pressure sensor 300 from damage and may transmit pulse pressure waves to the pressure sensor 300. The resiliently deformable cap 302 may be a pressurized bladder filled with air, some other fluid, or an elastomeric material. In some embodiments, the resiliently deformable cap 302 may be integrated with a resiliently deformable pad 308 to form a resiliently deformable pad-cap structure.

The embodiment depicted in FIG. 3 includes a rigid internal structure 306 and a plunger structure 304 coupled to the rigid internal structure 306. The plunger structure 304 is configured to transmit a pulse pressure wave from the rigid internal structure 306 to the pressure sensor 300. The pressure sensor 300 is directly or indirectly coupled to the plunger structure 304 on a side of the plunger structure 304 opposite the rigid internal structure 306. The resiliently deformable pad 308 covers the rigid internal structure 306 at least on a side of the rigid internal structure 306 opposite the plunger structure 304.

A cross-sectional view of a sensing device 400 is depicted in FIG. 4, according to an embodiment of the present disclosure. The sensing device 400 includes a pressure sensor 402, a rigid internal structure 404, and a resiliently deformable pad 406. The sensing device 400 further includes a plunger structure 408 coupled to the rigid internal structure 404. In the embodiment of FIG. 4, the plunger structure 408 is coupled directly to the pressure sensor 402. Coupling the plunger structure 408 directly to the pressure sensor 402 has the advantage of making the sensing device 400 more sensitive to pulse pressure waves received by the rigid internal structure 404 than if the pressure sensor 402 were indirectly coupled to the plunger structure 408, since pulse pressure waves undergo less damping between the rigid internal structure 404 and the pressure sensor 402.

However, the resilient deformable pad 406 may still protect the pressure sensor 402 from damage by damping forces exerted on it.

The plunger structure 408 may be oriented along an axis normal to a plane tangent to a skin contacting surface of the resiliently deformable pad 406 at a point of contact with skin 410 of a user. This orientation of the plunger structure 408 causes the pressure sensor 402 to be aligned with the component of the pulse pressure wave that travels outward from the skin 410 of the user.

The rigid internal structure 404 may be prestressed by the resiliently deformable pad 406 and may apply a baseline pressure to the pressure sensor 402. This prestress clay be applied by tensioning structures 412A and 412B that put tension on the resiliently deformable pad 406. The tension applied to the resiliently deformable pad 406 may cause the resiliently deformable pad 406 to, in turn, put pressure on the rigid internal structure 404. Via the rigid internal structure 404, pressure may be applied to the pressure sensor 402 so as to provide a constant biasing signal.

In another embodiment not shown, the resiliently deformable pad 406 may be divided into a top piece and a bottom piece. The top piece and the bottom piece may be attached to each other, for example with glue, in such a way that the rigid internal structure 404 is prestressed and a baseline pressure is applied to the pressure sensor 402.

A cross-sectional view of a sensing device 500 is depicted in FIG. 5, according to another embodiment of the present disclosure. The sensing device 500 of FIG. 5 differs from the sensing device 400 of FIG. 4 in that while the rigid internal structure 404 is coupled directly to the pressure sensor 402 by the plunger structure 408 in FIG. 4, a pressure sensor 502 is coupled indirectly to a rigid internal structure 504 in FIG 5. The sensing device 500 of FIG. 5 does not include a plunger structure. Instead, the pressure sensor 502 is coupled to a pressurized fluid 508 contained within the sensing device 500. The pressurized fluid 508 may be air, or may be some other fluid. The use of a pressurized fluid 508, rather than direct coupling between the pressure sensor 502 and a rigid internal structure 504, has the advantage of better protecting the pressure sensor 502. However, more of the force exerted by the pulse pressure wave on the sensing device 500 may be dissipated via damping in the pressurized fluid 508 than would be dissipated in an embodiment which direct coupling is used.

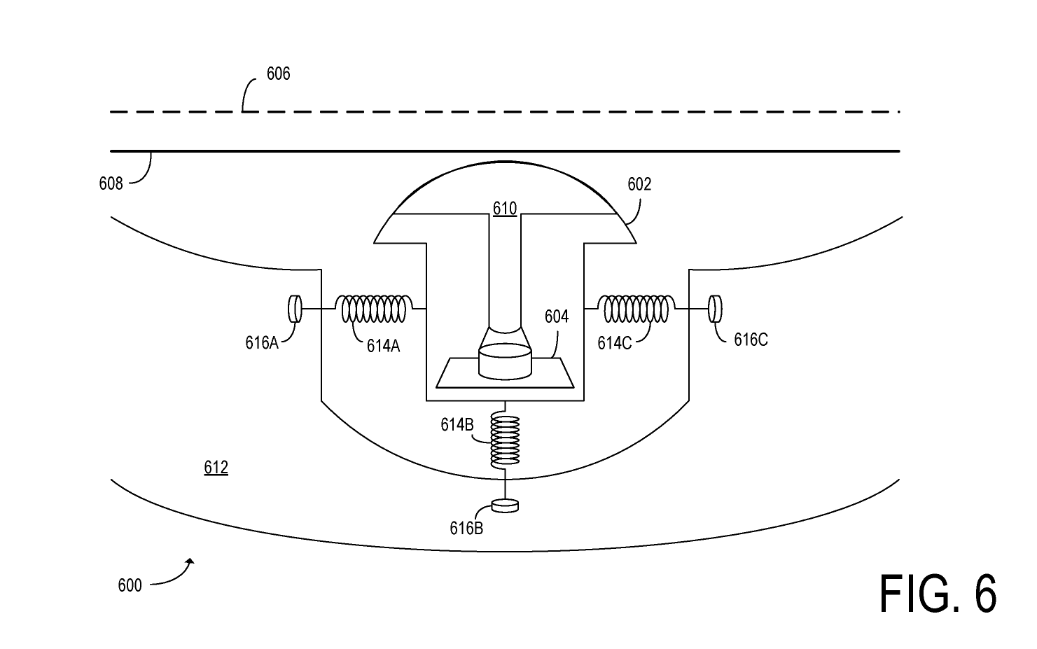

According to another embodiment of the present disclosure, depicted in cross section in FIG. 6, a wearable tonometer 600 is provided, including a sensing device 602. The sensing device 602 includes a pressure sensor 604 configured to measure a pulse pressure wave of blood pressure in an artery 606 of a user 608. The sensing device 602 further includes a resiliently deformable pad-cap structure 610 positioned between the pressure sensor 604 and skin of the user 608 proximate the artery 606. The resiliently deformable pad-cap structure 610 may consist of cloth, foam, an elastomeric material, or some other resiliently deformable material or combination of materials. In addition, the resiliently deformable pad-cap structure 610 may include a pressurized bladder filled with air, some other fluid, or an elastomeric material.

The resiliently deformable pad-cap structure 610 may be configured to applanate the skim of the user 608 such that the sensing device 602 contacts a wide enough area of skin on the user 608 that the wearable tonometer 600 may make an accurate blood pressure measurement without requiring precise placement. By distributing force exerted on the skin of the user 608 by the sensing device 602 over a larger area, the resiliently deformable pad-cap structure 610 may make the wearable tonometer 600 more comfortable to wear for a long period of time. Furthermore, the resiliently deformable pad-cap structure 610 may protect the pressure sensor 604 by damping forces exerted on the sensing device 602. Since the wearable tonometer 600 may be worn while the user 608 engages in a wide range of possible activities, it is useful for the resiliently deformable pad-cap structure 610 to protect the pressure sensor 604 and other components of the wearable tonometer 600 that may be fragile.

The wearable tonometer 600 includes a band 612 that holds the sensing device 602 in contact with the skin of the user 608. The wearable tonometer 600 further includes an adjustment mechanism configured to move the sensing device 602 relative to the band 612. In the embodiment of FIG. 6, the adjustment mechanism includes biasing members 614A, 614B, and 614C. Although three biasing members are depicted, other numbers of biasing members may be used. The adjustment mechanism is configured to move the sensing device 602 in a plane tangent to the skin of the user 608 while the position of the band 612 remains fixed. In the current embodiment, the biasing members 614A, 614B, and 614C are configured to move the sensing device 602 in a plane tangent to the skin of the user 608. For example, in response to a displacement of the sensing device 602 to the right relative to the band 612, the biasing members 614A, 614B, and 614C may exert forces in the direction of an equilibrium position to the left. Adjustments made by the adjustment mechanism to the position of the sensing device 602 may allow the sensing device 602 to remain in a location proximate the artery 606 of the user 608 when the wearable tonometer 600 is worn during activities that include movement. The adjustment mechanism may also make the sensing device 602 more comfortable for the user 608 by allowing movement of the sensing device 602 relative to the band 612 when the wrist of the user 608 bends.

In addition to moving the sensing device 602 in a plane tangent to the skin of the user 608, the adjustment mechanism may be further configured to move the sensing device 602 along an axis normal to the plane tangent to the skin of the user 608. For example, in response to a displacement of the sensing device 602 downward relative to the band 612 due to an ease in pressure exerted on the sensing device 602 by the skin of the user 608, the biasing members 614A, 614B, and 614C may exert forces in the direction of an equilibrium position upward. Adjusting the position of the sensing device 602 in this way decreases the applanation pressure and may make the wearable tonometer 600 more comfortable to wear, in comparison to embodiments that do not include a mechanism configured to adjust the applanation pressure. Additionally, in response to a displacement of the sensing device 602 upward relative to the band 612 due to an increase in pressure exerted on the sensing device 602 by the skin of the user 608, the biasing members 614A, 614B, and 614C may exert forces in the direction of an equilibrium position downward. Such an adjustment may allow the sensing device 602 to remain in contact with the skin of the user 608 when the band 612 is moved away from the skin.

In addition the biasing members 614A, 614B, and 614C, the adjustment mechanism of the wearable tonometer 600 includes electromagnetic actuators 616A, 616B, and 616C. Each electromagnetic actuator is configured to move its respective biasing member along an axis of that biasing member. The electromagnetic actuators 616A, 616B, and 616C may dynamically move the sensing device 602 in a plane tangent to the skin of the user 608 so that the sensing device 602 is in contact with the skin covering a surface artery 606 of the user 608. By moving the biasing members, the electromagnetic actuators may move the sensing device 602 in a plane tangent to the skin of the user 608 or along the axis normal to that plane. When the electromagnetic actuators 616A, 616B, and 616C are not moving their respective biasing members, the biasing members may passively adjust the position of the sensing device 602 as described above.

In some embodiments, the wearable tonometer 600 may perform intermittent blood pressure measurements rather than continuously measuring a pulse pressure wave of blood pressure in the artery 606 of the user 608. In such embodiments, the adjustment mechanism may be configured to receive a starting signal indicating a beginning of a blood pressure measurement, in response to receiving the starting signal, the adjustment mechanism may be configured to increase the applanation pressure of the sensing device 602 against the skin of the user 608. The adjustment mechanism may increase the applanation pressure by moving the biasing members 614A, 614B, and 614C using the electromagnetic actuators 616A, 616B, and 616C. The adjustment mechanism may be further configured to receive an ending signal indicating an ending of the blood pressure measurement. In response to receiving the ending signal, the adjustment mechanism may be configured to decrease the applanation pressure of the sensing device 602 against the skin of the user 608. By increasing the applanation pressure when blood pressure measurements are taken and holding the sensing device 602 in contact with the skin of the user 608 when measurements are not being taken, the adjustment mechanism may make the wearable tonometer 600 more comfortable for long-term use while still providing sufficient applanation pressure to allow for accurate blood pressure measurements when measurements are taken.

In some embodiments, the adjustment mechanism may be configured to ac just the applanation pressure of the sensing device 602 against the skin of the user 608 based at least in part on a pressure measurement made by the pressure sensor 604. For example, the pressure sensor 604 may detect that the applanation pressure of the sensing device 602 against the skin of the user 608 has dropped below some predetermined threshold, wherein blood pressure measurements made when the applanation pressure is below that threshold are unlikely to be accurate. In response to this detection, the adjustment mechanism may move the sensing device 602 so that the applanation pressure increases. Similarly, the pressure sensor 604 may detect that the applanation pressure has risen above a second predetermined threshold, wherein the wearable tonometer 600 is likely to cause discomfort when the applanation pressure is above that threshold. In response to this detection, the adjustment mechanism may move the sensing device 602 so that the applanation pressure decreases.

According to other embodiments not depicted, the wearable tonometer may include biasing members configured to move the sensing device in a plane tangent to the skin of the user or along the axis normal to that plane, but not include electromagnetic actuators. In such embodiments, the biasing members would have one end fixed in place in the sensing device and one end fixed in place in the band.

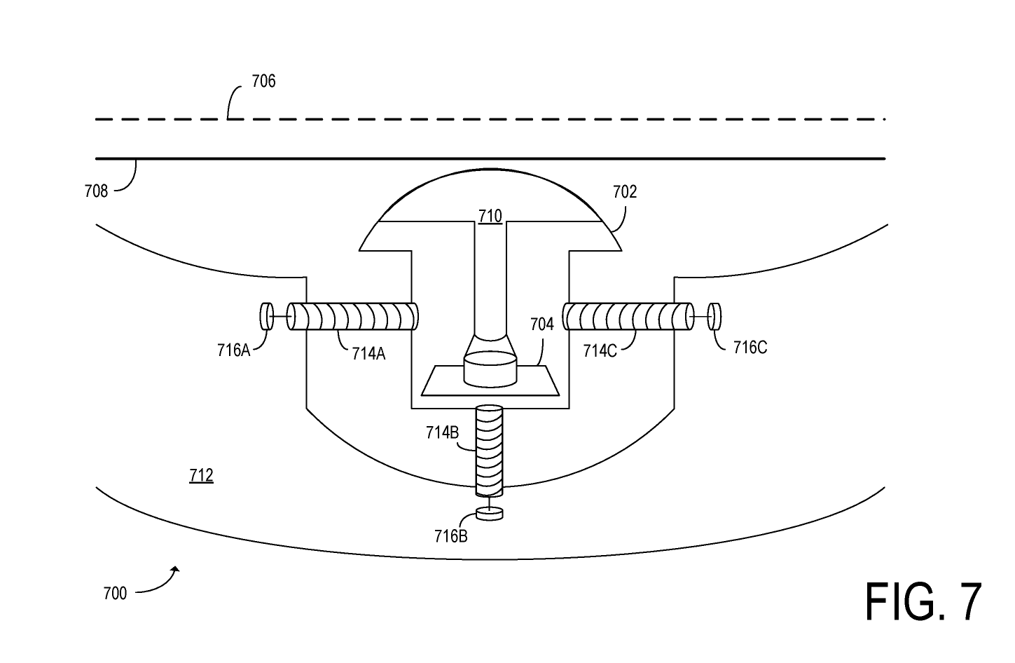

FIG. 7 shows an embodiment of a wearable tonometer 700 similar to that of FIG. 6, except that the adjustment mechanism includes screw members 714A, 714B, and 714C in place of biasing members. Although three screw members are depicted, other numbers of screw members may be used. The screw members 714A, 714B, and 714C are configured to be turned by micromotors 716A, 716B, and 716C respectively in order to adjust the position of a sensing device 702 relative to a hand 712. Adjusting the position of the sensing device 702 relative to the band 712 may include moving the sensing device 702 in a plane tangent to the skin of the user 708 or along the axis normal to that plane. By adjusting the position of the sensing device 702 relative to the band 712, the adjustment mechanism may move the sensing device 70 so that the resiliently deformable pad-cap structure 710 remains in contact with skin proximate a surface artery 706 of the user 708.

The adjustment mechanism may be configured to adjust an applanation pressure of the sensing device 702 against the skin of the user 708. As in the embodiment of FIG. 6, the adjustment mechanism of the wearable tonometer 700 of FIG. 7 may be configured to receive a starting signal indicating a beginning of a blood pressure measurement. In response to receiving the starting signal, the adjustment mechanism may increase the applanation pressure of the sensing device 702 against the skin of the user 708. The applanation pressure may be increased by turning at least one screw member using its micromotor. The adjustment mechanism may be further configured to receive an ending signal indicating an ending of the blood pressure measurement. In response to receiving the ending signal, the adjustment mechanism may be configured to decrease the applanation pressure of the sensing device 702 against the skin of the user 708.

The adjustment mechanism may be further configured to adjust the applanation pressure of the sensing device 702 against the skin of the user 708 based at least in part on a pressure measurement made by the pressure sensor 704. When the pressure measurement drops below some predetermined threshold, at least one micromotor may move at least one screw member so as to increase the applanation pressure, and when the pressure measurement rises above below some predetermined threshold, at least one micromotor may move at least one screw member so as to decrease the applanation pressure.

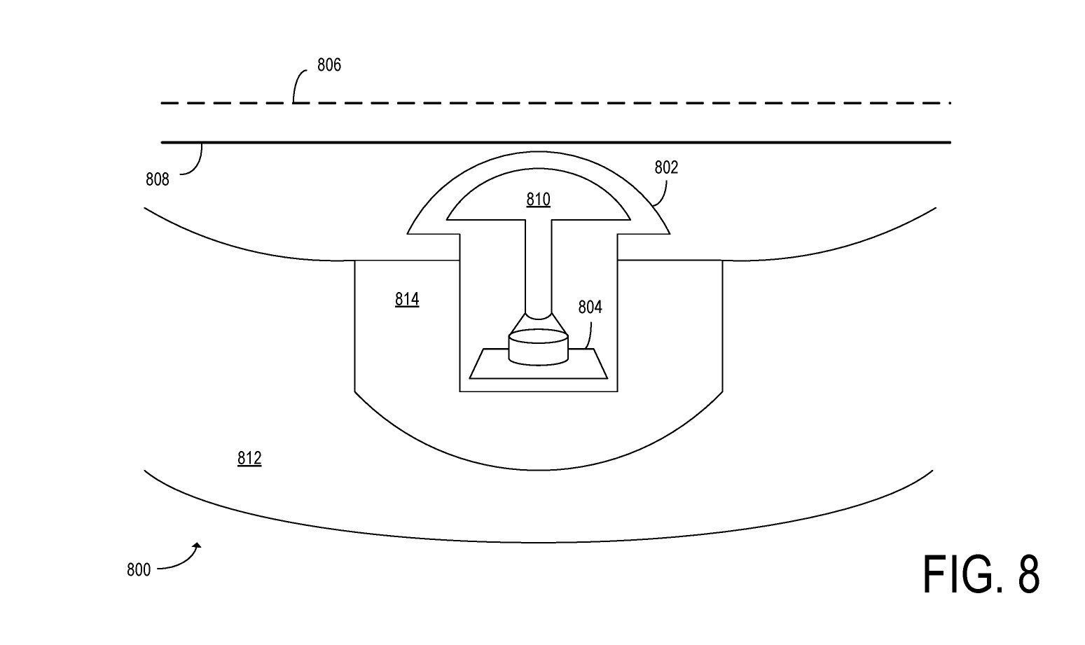

Another embodiment of the present disclosure is shown in FIG. 8. According to the embodiment depicted in FIG. 8, a wearable tonometer 800 is provided, including a sensing device 802. The sensing device 802 includes a pressure sensor 804 configured to measure a pulse pressure wave of blood pressure in an artery 806 of a user 808. The sensing device 802 further includes a resiliently deformable pad-cap structure 810 positioned between the pressure sensor 804 and skin of the user 808 proximate the artery 806. The wearable tonometer 800 also includes a band 812 that holds the sensing device 802 in contact with the skin of the user 808.

In the embodiment of FIG. 8, the pressure sensor 804 is coupled to a pressurized fluid 814 contained within the sensing device 802. The pressurized fluid 814 may be air, or may be some other fluid. When pressure is exerted on the sensing device 802, the pressurized fluid 814 exerts pressure in the opposite direction on the sensing device 802. The pressurized fluid 814 functions similarly to the biasing members 614A, 614B, and 614C in the embodiment of FIG. 6, in that it exerts a force on the sensing device 802 in a direction opposite that of a displacement from an equilibrium position. The pressurized fluid 814 may thus adjust applanation pressure of the sensing device 802 against the skin of the user 808.

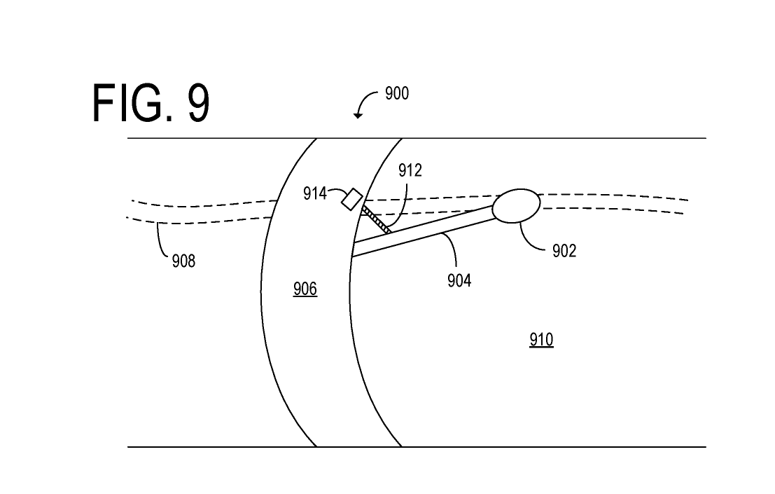

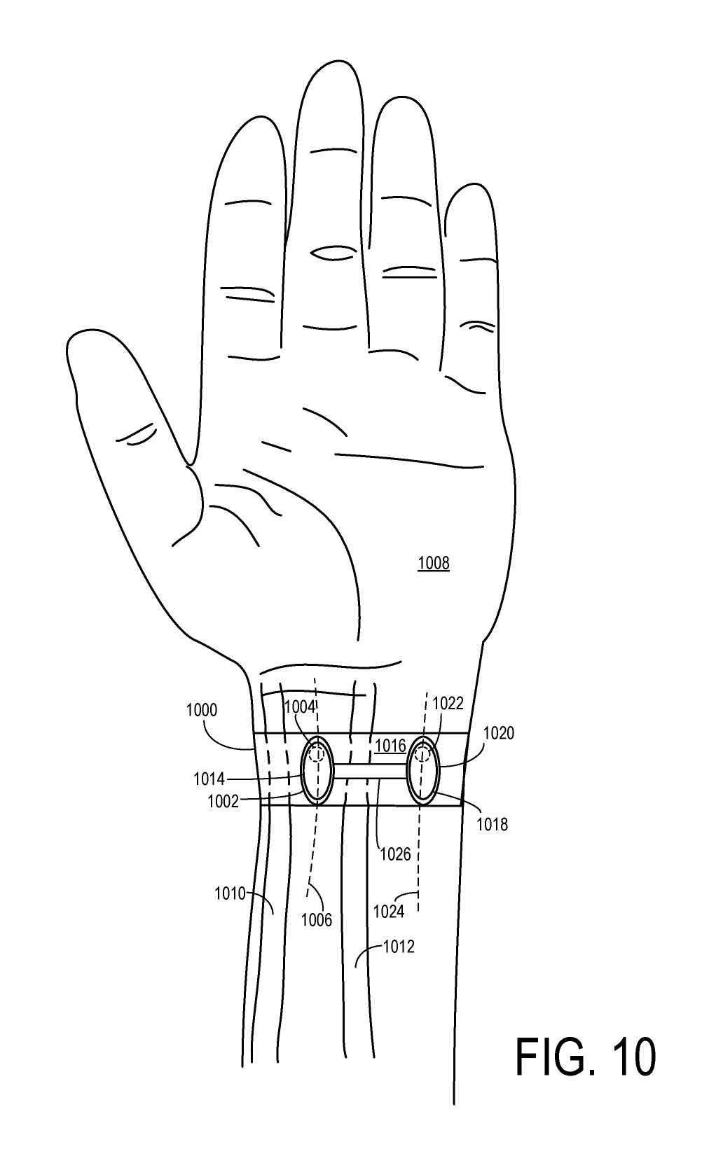

FIG. 9 shows another embodiment of the present disclosure. In FIG. 9, a wearable tonometer 900 is shown, including a sensing device 902. The wearable tonometer 900 further includes a cantilever 904 that is coupled on one end to the sensing device 902 and coupled on another end to a band 906. The cantilever 904 may be configured to move the sensing device 902 relative to the band 906 so that it is in contact with skin proximate an artery 908 of a user 910. The wearable tonometer 900 also includes a screw member 912 configured to move the cantilever 904, and a micromotor 914 configured to turn the screw member 912. Although the embodiment depicted in FIG. 9 only includes one screw member 912 and one micromotor 914, other numbers of screw members and micromotors may be used in other embodiments.

The micromotor 914 may be configured to move the cantilever 904 in a plane tangent to the skin of the user 910 while the position of the band 906 remains fixed, and/or in a plane perpendicular to that plane and parallel to the artery 908 of the user 910. The cantilever 904 may be dynamically moved in the plane tangent to the skin of the user 910 so that the sensing device 902 is in contact with the skin covering the artery 908 of the user 910.

According to another embodiment of the present disclosure, shown in FIG. 10, a wearable tonometer 1000 is provided, comprising a first sensing device 1002. The first sensing device 1002 includes a first pressure sensor 1004 configured to measure a pulse pressure wave of blood pressure in an artery 1006 of a user 1008. In the embodiment depicted in FIG. 10, the artery 1006 is a radial artery and the first sensing device 1002 sits between the radius 1010 and flexor carpi radialis tendon 1012 of the user 1008. The wearable tonometer 1000 may additionally or instead be configured to measure a pulse pressure wave of blood pressure in other arteries of the user 1008.

The first sensing device 1002 further includes a first resiliently deformable pad-cap structure 1014 positioned between the first pressure sensor 1004 and skin of the user 1008 proximate the artery 1006. The first resiliently deformable pad-cap structure 1014 is configured to apply an applanation pressure to the skin proximate the artery 1006 of the user 1008.

In addition, the first resiliently deformable pad-cap structure 1014 is configured to protect the first pressure sensor 1004 from damage. The first resiliently deformable pad-cap structure 1014 may consist of cloth, them, an elastomeric material, or some other resiliently deformable, material. or combination of materials. The wearable tonometer 1000 further includes a band 1016 that is configured to hold the first sensing device 1002 in contact with the skin of the user 1008.

The wearable tonometer 1000 of FIG. 10 further includes a second resiliently deformable pad-cap structure 1018 adjacent to skin of the user 1008. The use of a second resiliently deformable pad-cap structure 1018 as well as a first resiliently deformable pad-cap structure 1014 allows the applanation pressure exerted by the wearable tonometer 1000 on the skin of the user 1008 to be distributed over a larger surface. The second resiliently deformable pad-cap structure 1018 may also provide additional stability for the first sensing device 1002 to help keep it in place.

The second resiliently deformable pad-cap structure 1018 may be included in a second sensing device 1020. The second sensing device 1020 may include a second pressure sensor 1022 configured to measure a pulse pressure wave of blood pressure in an artery 1024 of the user 1008. In the embodiment depicted in FIG. 10, the artery for which the second pressure sensor 1022 is configured to measure a pulse pressure wave of blood pressure is an ulnar artery, while the first pressure sensor 1004 is configured to measure a pulse pressure wave of blood pressure in the radial artery. In other embodiments, the first pressure sensor 1004 and the second pressure sensor 1022 may be configured to measure pulse pressure waves of blood pressure in the same artery.

The wearable tonometer 1000 may be configured to determine whether the pulse pressure wave signal from the first pressure sensor 1004 or the second pressure sensor 1022 is higher in quality, based on, for example, noise in each of the measurements. The wearable tonometer 1000 may also be configured to average the signals from the pressure sensors, or to perform other operations that take the first and second pressure measurements as inputs and produce some output.

The wearable tonometer 1000 further includes a solid plate 1026 attached to the first resiliently deformable pad-cap structure 1014, the second resiliently deformable pad-cap structure 1018, and the band 1016. In the embodiment of FIG. 10, the solid plate 1026 bridges the flexor carpi radialis tendon 1012 of the user 1008, thus allowing the first sensing device 1002 and the second sensing device 1020 to remain in place relative to the skin of the user 1008 while the flexor carpi radialis tendon 1012 of the user 1008 moves. In other embodiments, the solid plate 1026 may bridge other tendons and allow those tendons to move while keeping the sensing devices in place.

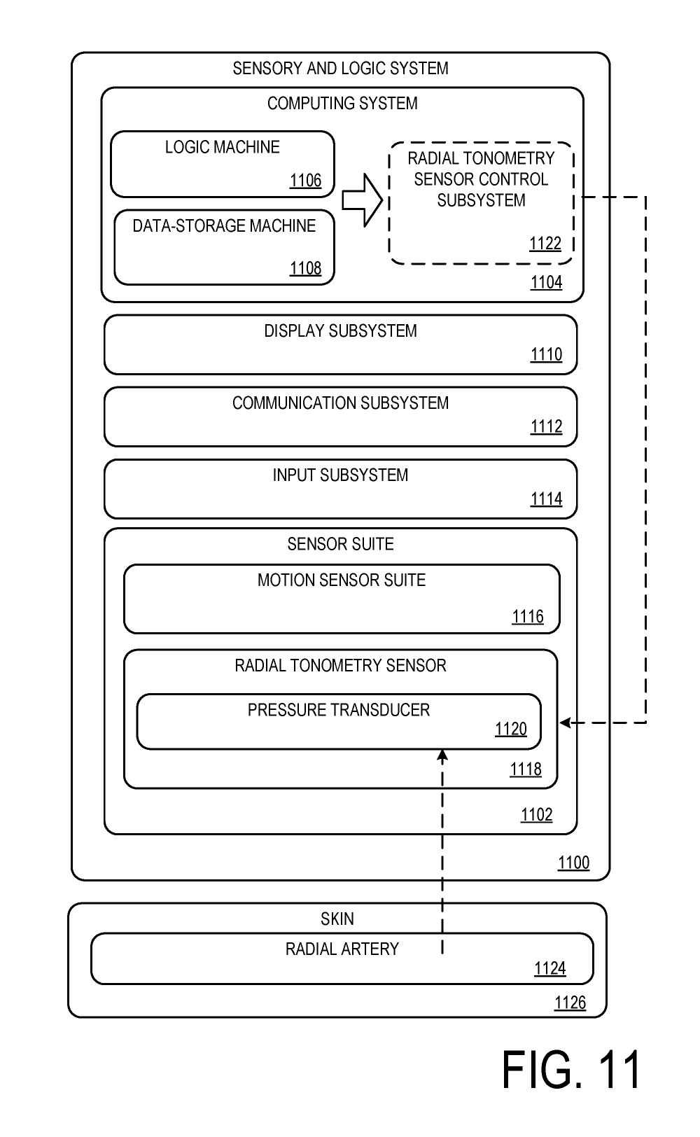

FIG. 11 schematically shows a form-agnostic sensory and logic system 1100 for use with a wearable tonometer. The sensory and logic system 1100 includes a sensor suite 1102 operatively coupled to a computing system 1104. The computing system 1104 includes a logic machine 1106 and a data-storage machine 1108. The computing system 1104 is operatively coupled to a display subsystem 1110, a communication subsystem 1112, an input subsystem 1114, and/or other components not shown in FIG. 11.

Logic machine 1106 includes one or more physical devices configured to execute instructions. The logic machine 1106 may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

Logic machine 1106 may include one or more processors configured to execute software instructions. Additionally or alternatively, the logic machine 1106 may include one re hardware or firmware logic machines configured to execute hardware or firmware instructions. Processors of the logic machine 1106 may be single-core or multi-core, and the instructions executed thereon may be configured for sequential, parallel, and/or distributed processing. Individual components of a logic machine optionally may be distributed among two or more separate devices, which may be remotely located and/or configured for coordinated processing. Aspects of a logic machine may be virtualized and executed by remotely accessible, networked computing devices in a cloud-computing configuration.

Data-storage machine 1108 includes one or more physical devices configured to hold instructions executable by logic machine 1106 to implement the methods and processes described herein. When such methods and processes are implemented, the state of the data-storage machine 1108 may be transformed to hold different data. The data-storage machine 1108 may include removable and/or built-in devices; it may include optical memory (e.g., CD, DVD HD-DVD, Blu-Ray Disc, etc.), semiconductor memory (e.g., RAM, EPROM, EEPROM, etc.), and/or magnetic memory hard-disk drive, floppy-disk drive, tape drive, MRAM, etc.), among others. The data-storage machine 1108 may include volatile, nonvolatile, dynamic, static, read/write, read-only, random-access, sequential-access, location-addressable, file-addressable, and/or content-addressable devices.

Data-storage machine 1108 includes one more physical devices. However, aspects of the instructions described herein alternatively may be propagated by a communication medium (e.g., an electromagnetic signal, an optical signal, etc.) that is not held by a physical device for a finite duration.

Aspects of logic machine 1106 and data-storage machine 1108 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

Display subsystem 1110 may be used to present a visual representation of data held by data-storage machine 1108. This visual representation may take the form of a graphical user interface (GUI). As the herein described methods and processes change the data held by the data-storage machine 1108, and thus transform the state of the data-storage machine, 1108, the state of display subsystem 1110 may likewise be transformed to visually represent changes in the underlying data. Display subsystem 1110 may include one or more display subsystem devices utilizing virtually any type of technology. Such display subsystem devices may be combined with logic machine 1106 and/or data-storage machine 1108 in a shared enclosure ,or such display subsystem devices may be peripheral display subsystem devices.

Communication subsystem 1112 may be configured to communicatively couple computing system 1104 to one or more other computing devices. The communication subsystem 1112 may include wired and/or wireless communication devices compatible with one or more different communication protocols. As non-limiting examples, the communication subsystem 1112 may be configured for communication via a wireless telephone network, a local- or wide-area network, and/or the Internet.

Input subsystem 1114 may comprise or interface with one or more user-input devices such as a keyboard, touch screen, button, dial, joystick, or switch. In some embodiments, the input subsystem 1114 may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition.

Sensor suite 1102 may include one or more different sensors for example, radial tonometry sensor 1118, a touch-screen sensor, push-button sensor, microphone, visible-light sensor, ultraviolet sensor, ambient-temperature sensor, contact sensors, and/or GPS receiver as described above with reference to FIG. 1. Sensor suite 1102 may include motion sensor suite 1116. Motion sensor suite 1116 may include one or more of an accelerometer, gyroscope, magnetometer, or other suitable motion detectors.

As described herein, radial tonometry sensor 1118 may include pressure transducer 1120. Computing system 1104 may include radial tonometry sensor control subsystem 1122, which may be communicatively coupled to logic machine 1106 and data-storage machine 1108. Pressure transducer 1120 may comprise one or more piezo-resistive sensors configured to provide absolute pressure signals to computing system 1104 via an analog-to-digital converter. Pressure transducer 1120 may be configured to transduce pressure waves from the artery 1124 through the skin 1126 of the user.

Radial tonometry sensor control subsystem 1122 may further process the raw signals to determine heart rate, blood pressure, caloric expenditures, etc. Processed signals may be stored and output via computing system 1104. Control signals sent to radial tonometry sensor 1118 may be based on signals received from pressure transducer 1120, signals derived from sensor suite 1102, information stored in data-storage machine 1108, input received from communication subsystem 1112, input received from input subsystem 1114, etc.

The following paragraphs provide additional support for the claims of the subject application. According to one aspect of the present disclosure, a wearable tonometer is provided, comprising a sensing device. The sensing device may include a pressure sensor configured to measure a pulse pressure wave of blood pressure in an artery of a user. The sensing device may further include a resiliently deformable pad positioned on a sensing surface side of the pressure sensor and configured to contact skin of the user proximate the artery. The sensing device may further include a rigid internal structure positioned at least partially within the resiliently deformable pad and configured to transmit the pulse pressure wave on its way to the pressure sensor. The wearable tonometer may further include a band that holds the sensing device in contact with the skin of the user.

According to this aspect, the wearable tonometer may further include a plunger structure coupled to the rigid internal structure. The pressure sensor may be directly or indirectly coupled to the plunger structure on a side of the plunger structure opposite the rigid internal structure.

According to this aspect, the plunger structure may be coupled directly to the pressure sensor.

According to this aspect, the resiliently deformable pad may cover the rigid internal structure at least on a side of the rigid internal structure opposite the plunger structure.

According to this aspect, the plunger structure may be oriented along an axis normal to a plane tangent to a skin contacting surface of the resiliently deformable pad at a point of contact with the skin of the user.

According to this aspect, the pressure sensor may be coupled to a pressurized fluid contained within the sensing device.

According to this aspect, the rigid internal structure may be prestressed by the resiliently deformable pad and may apply a baseline pressure to the pressure sensor.

According to this aspect, the resiliently deformable pad may cover the entire sensing device.

According to this aspect, the resiliently deformable pad may have a convex skin-contacting surface that is elongated along a longitudinal axis.

According to this aspect, the rigid internal structure may have a plate-like shape that is elongated along the longitudinal axis.

According to another aspect of the present disclosure, a wearable tonometer is provided, comprising a sensing device. The sensing device may include a pressure sensor configured to measure a pulse pressure wave of blood pressure in au artery of a user. The sensing device may further include a resiliently deformable pad-cap structure positioned between the pressure sensor and skin of the user proximate the artery. The wearable tonometer may further include a band that may hold the sensing device in contact with the skin of the user. The wearable tonometer may further include an adjustment mechanism configured to move the sensing device relative to the band. The adjustment mechanism may be configured to move the sensing device in a plane tangent to the skin of the user while the position of the band remains fixed.

According to this aspect, the adjustment mechanism may be further configured to adjust an applanation pressure of the sensing device against the skin of the user.

According to this aspect, the adjustment mechanism may be further configured to receive a starting signal indicating a beginning of a blood pressure measurement. In response to receiving the starting the adjustment mechanism may be configured to increase the applanation pressure of the sensing device against the skin of the user. The adjustment mechanism may be further configured to receive an ending signal indicating an ending of the blood pressure measurement. In response to receiving the ending signal, the adjustment mechanism may be configured to decrease the applanation pressure of the sensing device against the skin of the user.

According to this aspect, the adjustment mechanism may be configured to adjust the applanation pressure of the sensing device against the skin of the user based at least in part on a pressure measurement made by the pressure sensor.

According to this aspect, the sensing device may be dynamically moved in a plane tangent to the skin of the user so that the sensing device is in contact with the skin covering a surface artery of the user.

According to this aspect, the adjustment mechanism may include a biasing member or screw member.

According to this aspect, the pressure sensor may be coupled to a pressurized fluid contained within the sensing device.

According to this aspect, the adjustment mechanism may include a cantilever that is coupled on one end to the sensing device and coupled on another end to the hand

According to another aspect of the present disclosure, a wearable tonometer is provided, comprising a first sensing device. The first sensing device may include a first pressure sensor configured to measure a pulse pressure wave of blood pressure in an artery of a user. The first sensing device may further include a first resiliently deformable pad-cap structure positioned between the first pressure sensor and skin of the user proximate the artery. The wearable tonometer may further include a band that holds the first sensing device in contact with the skin of the user. The wearable tonometer may further include a second resiliently deformable pad-cap structure adjacent to skin of the user. The wearable tonometer may further include a solid plate attached to the first resiliently deformable pad-cap structure, the second resiliently deformable pad-cap structure, and the band.

According to this aspect, the wearable tonometer may further include a second sensing device, wherein the second sensing device includes a second pressure sensor configured to measure a pulse pressure wave of blood pressure in an artery of the user. The wearable tonometer may further include the second resiliently deformable pad-cap structure.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A wearable tonometer, comprising:
a sensing device, wherein the sensing device includes:
a pressure sensor configured to measure a pulse pressure wave of blood pressure in an artery of a user;
a resiliently deformable pad positioned on a sensing surface side of the pressure sensor and configured to contact skin of the user proximate the artery;
a rigid internal structure positioned at least partially within the resiliently deformable pad and configured to transmit the pulse pressure wave on its way to the pressure sensor; and
a band that holds the sensing device in contact with the skin of the user.

2. The wearable tonometer of claim 1, further comprising a plunger structure coupled to the rigid internal structure, wherein the pressure sensor is directly or indirectly coupled to the plunger structure on a side of the plunger structure opposite the rigid internal structure.

3. The wearable tonometer of claim 2, wherein the plunger structure is coupled directly to the pressure sensor.

4. The wearable tonometer of claim 2, wherein the resiliently deformable pad covers the rigid internal structure at least on a side of the rigid internal structure opposite the plunger structure.

5. The wearable tonometer of claim 2, wherein the plunger structure is oriented along an axis normal to a plane tangent to a skin contacting surface of the resiliently deformable pad at a point of contact with the skin of the user.

6. The wearable tonometer of claim 1, wherein the pressure sensor is coupled to a pressurized fluid contained within the sensing device.

7. The wearable tonometer of claim 1, wherein the rigid internal structure is prestressed by the resiliently deformable pad and applies a baseline pressure to the pressure sensor.

8. The wearable tonometer of claim 1, wherein the resiliently deformable pad covers the entire sensing device.

9. The wearable tonometer of claim 1, wherein the resiliently deformable pad has a convex skin-contacting surface that is elongated along longitudinal axis.

10. The wearable tonometer of claim 9, wherein the rigid structure has a plate-like shape that is elongated along the longitudinal axis.

11. A wearable tonometer, comprising:
a sensing device, wherein the sensing device includes:
a pressure sensor configured to measure a pulse pressure wave of blood pressure in an artery of a user; and
a resiliently deformable pad-cap structure positioned between the pressure sensor and skin of the user proximate the artery;
a band that holds the sensing device in contact with the skin of the user; and
an adjustment mechanism configured to move the sensing device relative to the band, wherein the adjustment mechanism is configured to move the sensing device in a plane tangent to the skit of the user while the position of the band remains fixed.

12. The wearable tonometer of claim 11, wherein the adjustment mechanism is further configured to adjust an applanation pressure of the sensing device against the skin of the user.

13. The wearable tonometer of claim 12, wherein the adjustment mechanism is further configured to:
receive a starting signal indicating a beginning of a blood pressure measurement;
in response to receiving the starting signal, increase the applanation pressure of the sensing device against the skin of the user;
receive an ending signal indicating an ending of the blood pressure measurement; and
in response to receiving the ending signal, decrease the applanation pressure of the sensing device against the skin of the user.

14. The wearable tonometer of claim 12, wherein the adjustment mechanism is configured to adjust the applanation pressure of the sensing device against the skin of the user based at least in part on a pressure measurement made by the pressure sensor.

15. The wearable tonometer claim 11, wherein the sensing device is dynamically moved in a plane tangent to the skin of the user so that the sensing device is in contact with the skin covering a surface artery of the user.

16. The wearable tonometer of claim 11, wherein the adjustment mechanism includes a biasing member or screw member.

17. The wearable tonometer of claim 11, wherein the pressure sensor is coupled to a pressurized fluid contained within the sensing device.

18. The wearable tonometer of claim 11, wherein the adjustment mechanism includes a cantilever that is coupled on one end to the sensing device and coupled on another end to the band.

19. A wearable tonometer, comprising:
a first sensing device, wherein the first sensing device includes:
first pressure sensor configured to measure a pulse pressure wave of blood pressure in an artery of a user;
a first resiliently deformable pad-cap structure positioned between the first pressure sensor and skin of the user proximate the artery;
a band that holds the first sensing device in contact with the skin of the user;
a second resiliently deformable pad-cap structure adjacent to skin of the user; and
a solid plate attached to the first resiliently deformable pad-cap structure, the second resiliently deformable pad-cap structure, and the band.

20. The wearable tonometer of claim 19, further comprising a second sensing device, wherein the second sensing device includes:
a second pressure sensor configured to measure a pulse pressure wave of blood pressure in an artery of the user; and
the second resiliently deformable pad-cap structure.
